(19) United States
(12) Patent Application Publication
(10) Pub. No.: US 2017/0086899 A1
(43) Pub. Date: Mar. 30, 2017

(54) Method for Cryoablating Invasive Breast Carcinoma

(71) Applicant: SANARUS TECHNOLOGIES, INC., Pleasanton, CA (US)

(72) Inventors: Mathew J. Nalipinski, Pleasanton, CA (US); Lisa Henry, Pleasanton, CA (US); Alan Marquardt, Pleasanton, CA (US)

(73) Assignee: SANARUS TECHNOLOGIES, INC., Pleasanton, CA (US)

(21) Appl. No.: 14/870,852

(22) Filed: Sep. 30, 2015

(51) Int. Cl. A61B 18/02; A61B 8/08

(52) U.S. Cl. CPC A61B 18/02; A61B 8/0841; A61B 8/085; A61B 2018/00333

(57) ABSTRACT

A method of treating cancerous lesions with optimized freezing periods at a single freezing temperature and minimal thawing time between freezing periods.

### FIELD OF THE INVENTIONS

The inventions described below relate to the field of cryosurgery and the treatment of breast cancer.

### BACKGROUND OF THE INVENTIONS

The methods described below provide for optimal treatment of invasive breast carcinoma. In our prior patents, we disclosed methods and systems for treating fibroadenomas in the breast of female patients. Our experience with fibroadenoma patients has led to the development of the new procedure described below which applies cryoablation therapy as a nonoperative approach to the treatment of primary breast tumors. The new procedure is simple, readily available for use with existing cryotherapy devices and is less painless to the patient for treatment of cancer.

### SUMMARY

The methods and systems described below provide for effective cryoablation of target cancerous lesions and a margin of tissue analogous to the surgical margins of a lumpectomy. Cryoablation is performed with a treatment regimen including two freeze cycles with an intervening passive warming period, without an intervening low-power freeze cycle. When accomplished with commercially available cryoprobes such as our Visica® argon gas cryoprobes system or our Visica II™ liquid nitrogen cryoprobes system, the method entails a period of freezing, followed by a period of passive warming, followed by a repetition of these steps, and optionally followed by a warming cycle to speed removal of the cryoprobe from the iceball.

To achieve the coincident iceball, the cryoprobes are adapted to achieve an iceball, which roughly matches the typical shape of a target lesion. The cryoprobe tip is pushed into and through the target lesion so that its distal tip protrudes from the far boundary of the target lesion. The cryoprobe is then operated to create an iceball operated for a period of time, which depends on the size of the lesion, necessary to create an iceball engulfing the lesion. The treatment is achieved with two freezing periods and with a passive warming/thawing period between the two freezing periods that is determined empirically to achieve warming sufficient to exhaust known warming cell-death mechanisms. Measurements are obtained after each freezing period to ensure that sufficient margins have been achieved. In clinical practice, it depends on the length of time a cryoprobe is operated and its duty cycle, and this in turn depends on the cooling power of the cryoprobes, the shape of the iceball, and the location of the cryoprobe tip relative to the lesion.

### BRIEF DESCRIPTION OF THE DRAWINGS

FIG. 1 illustrates the cryosurgical procedure for treating benign tumors in the breast.

FIG. 2 illustrates the geometry of a typical fibroadenoma and the desired iceball used to ablate the fibroadenoma.

FIG. 3 illustrates a lesion and relevant dimensions to perform calculations to ensure proper placement of the probe through the lesion.

### DETAILED DESCRIPTION OF THE DRAWINGS

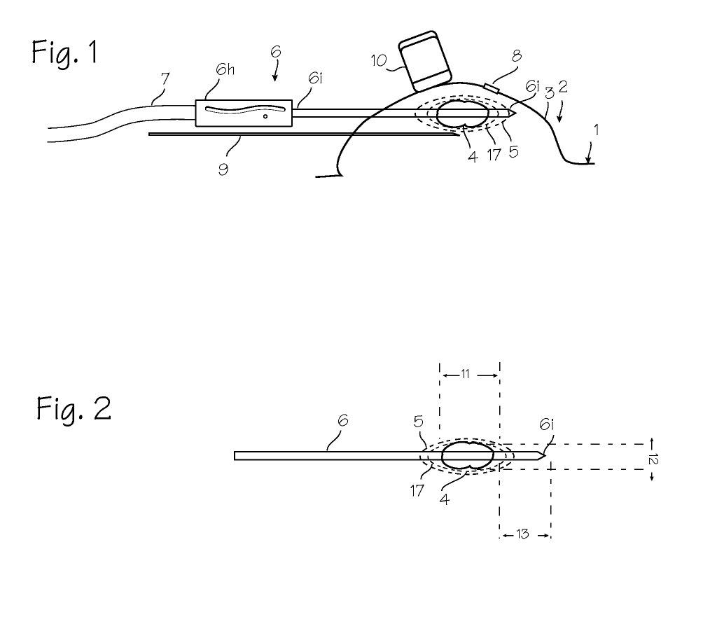

FIG. 1 illustrates the cryosurgical procedure for treating invasive breast carcinoma. The patient 1 and the patient's breast 2 and skin 3 of the breast are shown schematically. The lesion 4 is located within the breast, surrounded by soft tissue and fatty tissue. The lesion is a well-defined, mass ranging in size from less than 1 to 2.0 cm in diameter. The purpose of the procedure is to form an iceball 5 (the frozen mass of breast tissue) around the lesion, after which the natural healing processes of the body will result in resorption of the tumor by the patient's body. The iceball is formed with a cryoprobe 6, which, as illustrated, includes a handle portion 6h and an insertion portion 6i which is inserted through the skin and intervening breast tissue into the lesion, so that the distal tip extends through the lesion. A cryogen supply hose 7 is attached to the cryoprobe and serves to supply liquid cryogen or high-pressure gas to the cryoprobe. The cryoprobe may include a temperature sensor, which directly or indirectly measures the temperature of the cryoprobe. An external temperature sensor 8 may be used during the surgery to monitor skin temperature, so that surgeons can avoid causing frost-bite on the patient's skin. An internal temperature sensor 9 may be inserted into the breast to monitor the creation and maintenance of the iceball or the approximate location of the −20° C. isotherm. An ultrasound probe 10 is used during the procedure to visualize the formation, growth, and melting of the iceball that is formed within the breast when the cryoprobe is energized. (The iceball is highly echogenic, so that its formation is very clearly visualized. The image of the iceball is displayed on a display screen provided with the ultrasound probe.) The ultrasound probe is also used to measure maximum lesion width dimensions between freeze cycles in order to ensure appropriate treated margins of the lesion.

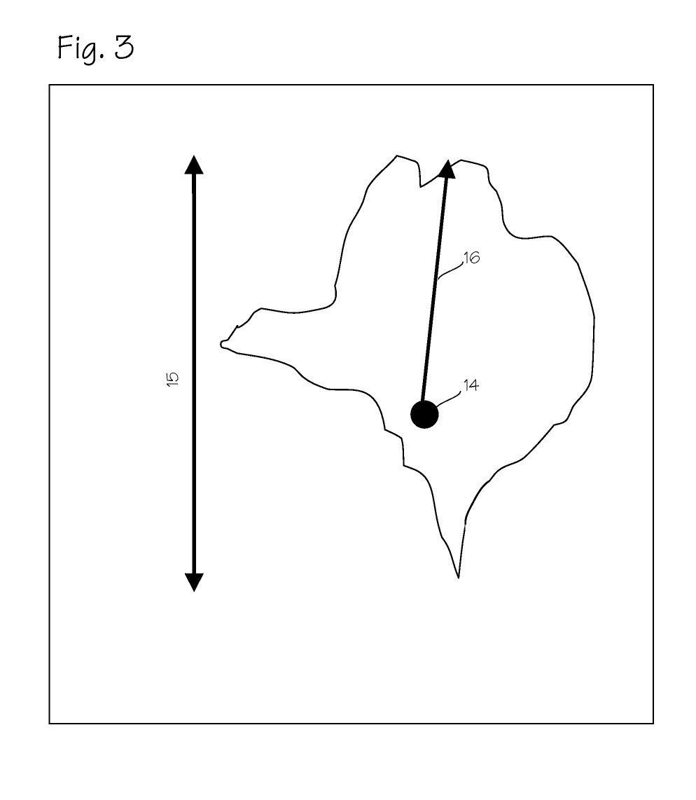

FIG. 2 illustrates the geometry of a typical tumor and the desired iceball used to ablate the lesion. The common lesion 4 is typically oblong, characterized by a major axis 11 and a minor axis 12. The lesion may range in size from less than 1 cm along the major axis to 2 cm along the major axis. The lesion may also range from less than 1 cm along the minor axis to 1.5 cm along the minor axis. The cryoprobe 6 has been designed to create an oblong iceball which matches the typical shape of a target lesion. The cryoprobe is inserted percutaneously through a small incision that is approximately 3-5 mm and pushed into the lesion so that the distal tip 6d protrudes past the far boundary (the distal boundary in relation to the cryoprobe) by a length 13. The cryoprobe is manipulated under real-time ultrasound guidance and the probe tip advanced through the center of the malignant lesion such that the length of the probe shaft penetrates and is centered in the longest imaged lesion plane measured. Proper probe placement is documented with longitudinal and transverse sonographic imaging. When placed properly through the lesion, the tumor will measure 1.5 cm or less in the minor axis and the maximum radius (shown in FIG. 3) from the probe shaft to the outermost edge (farthest tip from probe shaft tip 14) of the target lesion will be less than 0.75 cm.

FIG. 3 illustrates a lesion and relevant dimensions to perform calculations to ensure proper placement of the probe through the lesion. The maximum lesion width 15 is calculated at twice the maximum radius 16 as measured above. If the calculated maximum lesion width dimension to be treated (minor axis to probe within the target lesion) is greater than 1.5 cm., the probe must be relocated more central to the middle of the lesion. The length of distal protrusion 13 may be varied according the size of the lesion, to place the center of iceball near the center of the lesion. The calculated lesion width dimension is used to apply the treatment algorithm listed below if the calculated maximum lesion width dimension is greater than or equal to the largest imaged pre-biopsy lesion width of either mammogram, ultrasound or MRI. If the largest imaged pre-biopsy lesion width on either mammogram, ultrasound or MRI is larger than the calculated lesion width dimension imaged at the time of cryoablation, the pre-biopsy lesion width is used to determine which algorithm from the table below should be used. The iceball tends to form first at a known position probe, and then grow proximally and distally along the probe, but it is currently easier to visualize the distal protrusion than it is to visualize the coincidence of the probe's expected iceball and the center of the lesion. The iceball 5 is coldest at its center (at the surface of the cryoprobe). The 0° C. isotherm represents edge of the iceball (the boundary between frozen tissue and un-frozen tissue), while the −20° C. isotherm 17 lies within the iceball. The −20° C. isotherm is the boundary of that part of the iceball with is at or below −20° C. We want the volume of ice encompassed by the −20° C. isotherm to encompass the lesion.

The cryoprobe used for the procedure may be our Visica 2™ 3.4 mm cryoprobe (which uses liquid nitrogen), our Visica® cryoprobe (which uses argon gas and a Joule-Thomson cryostat), or other commercially available cryoprobes. Our Visica 2™ cryoprobe and system are described in detail in our prior U.S. Pat. No. 7,976,538, issued Jul. 12, 2011 and application Ser. No. 11/318,142 filed Dec. 23, 2005 and Ser. No. 11/406,547 filed Apr. 18, 2006 (212/846) and their corresponding PCT application PCT/US06/48863 filed Dec. 22, 2006, the entirety of each being incorporated by reference. The treatment system uses a closed system to circulate liquid nitrogen within the probe tip creating sub-freezing temperatures that result in precision cryoablation of the intended tissue target. The target lesion is first appropriately identified and then the probe is placed under ultrasound guidance into the enter of the lesion and cryoablated according to a predetermined freeze algorithm consisting of a freeze cycle followed by a thaw cycle, followed by a final freeze cycle. The probe is then warmed by an internal electrical resistance heater and removed from the patient.

This method of treating cancerous legions described below provides for fast treatment without loss of effectiveness. Once situated and positioned properly relative to the target lesion, the cryoprobe is operated for two cycles of high-power freezing, with a passive warming period interposed between the cycles and a warming period provided after the second freezing cycle, without any intervening low-power freezing periods. The periods of high-power freezing are selected depending on the size of the lesion and expected time for the cryoprobe to grow an iceball with a −20° C. isotherm. The period of passive warming between freezing periods are limited to the period necessary to allow substantial completion of known cell-death mechanisms which occur during warming. With experimentation, we have empirically determined the following freeze periods for lesions of various sizes using the liquid nitrogen cryoprobes of our Visica 2™ system:

| Major Axis (cm) | 0-0.9 | 1.0-1.5 |
|---|---|---|
| 1^{st} Freeze | 6 m | 8 m |
| Warm | 10 m | 10 m |
| 2^{nd} Freeze | 6 m | 8 m |
| Total Time | 22 m | 26 m |

As indicated in the table, a lesion with a major axis smaller than 1 cm is treated with two freezing cycles consisting each of 6 minutes of freezing (engulfing the mass in a −20° C. isotherm), with 10 minutes of passive warming between the freezing cycles. A lesion with a major axis of 1 to 1.5 cm diameter is treated by two cycles of freezing, each consisting of 8 minutes of freezing, with 10 minutes of passive warming between the cycles. It is necessary to ensure that the iceball diameter exceeds the calculated maximum lesion width dimension by 10 mm on all sides via ultrasound after each freezing cycle. It the 10 mm margins are not met, the freeze time is exceeded to ensure the iceball diameter exceeds the 10 mm requirement.

Thus, the method entails operating the cryoprobe for a first cooling period to create an iceball having a −20° C. isotherm defining a volume engulfing the lesion, such that the −20° C. isotherm is substantially coincident with an outer margin of the lesion along an axis of the lesion (preferably matching the minor axis of the iceball to the minor axis of the lesion) and thereafter, without substantial delay, ceasing operation of the cryoprobe for a warming period limited to the time necessary to allow the iceball to warm to 0° C. (without thawing) and allow completion of warming cell death mechanism, and thereafter, again without substantial delay, operating the cryoprobe for a second cooling period to create an iceball having a −20° C. isotherm defining a volume engulfing the lesion such that the −20° C. isotherm is substantially coincident with an outer margin of the lesion along an axis of the lesion, and thereafter, again without substantial delay, ceasing cooling operation of the cryoprobe and allowing or causing the cryoprobe to warm as necessary to remove the cryoprobe. Also, the warming necessary to remove the cryoprobe may be augmented by application of heat through the cryoprobe through any suitable active warming mechanism.

The cooling periods are preferably predetermined, in the sense that they are determined empirically based on the typical time required to create an iceball having a −20° C. isotherm defining a volume engulfing a typical lesion of the same approximate size as the lesion to be treated with a cryoprobe of similar design to the cryoprobe used to treat the lesion. The second cooling period may be determined empirically, based on the typical time required to create an iceball having a −20° C. isotherm defining a volume engulfing a typical lesion of the same approximate size as the lesion to be treated (in which case it is equivalent to the first cooling period), or based on the typical time required to cool the warmed iceball to re-create an iceball having a −20° C. isotherm (in which case it may be substantially shorter than the first cooling period, because it starts from a frozen state). In use, achievement of the appropriate size iceball is confirmed between freezing periods by ultrasound. Specifically, after the first freezing period, the iceball length and width are determined to ensure that the iceball diameter exceeds the calculated maximum lesion width dimension by 10 mm on all sides as determined by the ultrasound. If the 10 mm benchmark is not achieved, the first freezing time is extended. At the end of the second freezing period, the iceball length and width are measured again to ensure that iceball diameter exceeds the calculated maximum lesion width dimension by 10 mm on all sides as determined by the ultrasound. If the 10 mm benchmark is not achieved, the second freezing time is extended. Because cryoprobes of different design have different efficiencies and cooling powers, the predetermined cycle times may vary with the design of the cryoprobe and the operating mode of the cryoprobe.

These time periods may be varied to accomplish other regimens falling under the general description of two freezing cycles comprising creation of an iceball having a −20° C. isotherm substantially engulfing or coincident with the lesion, with a warming period between the freezing cycles. It is specifically contemplated that they be adjusted to account for cryoprobes of differing cooling power or cryoprobes from different manufacturers, and that the lesion size ranges be condensed or expanded as clinical experience dictates.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A method of treating a cancerous lesion in the breast of a patient, said method comprising:
providing a cryoprobe having a probe shaft to treat lesion, said cryoprobe characterized by a distal end and a proximal end and a longitudinal axis, said distal end being adapted for insertion into the lesion, said cryoprobe being operable to create an iceball;
determining the maximum lesion width by visualizing the lesion in a major axis relative to the cryoprobe shaft;
determining the major axis and the minor axis of the lesion to be treated, and inserting the distal end of the cryoprobe into said lesion such that the longitudinal axis is substantially aligned with the primary axis;
operating the cryoprobe for a first cooling period limited to the approximate time necessary to create an iceball extending at least 10 mm in all directions from the maximum lesion width and thereafter immediately ceasing operation of the cryoprobe for a warming period limited to the approximate time necessary to passively warm the iceball to 0° C. and allow substantial completion of warming cell death mechanisms, and immediately thereafter operating the cryoprobe for a second cooling period and thereafter ceasing cooling operation of the cryoprobe;
withdrawing the cryoprobe from the lesion.

2. The method of claim 2 wherein, in the step of operating the cryoprobe for a second cooling period, the second cooling period is operated for a period equivalent to the first cooling period.

3. The method of claim 1 wherein the warming period is determined empirically, based on the typical time required to warm an iceball of the size created in the first cooling period, within the body, to 0° C. without thawing the iceball.

4. A method of treating a cancerous lesion in the breast of a patient, said method comprising:
providing a cryoprobe to treat the lesion, said cryoprobe characterized by a probe shaft having a distal end and a proximal end and a longitudinal axis, said distal end adapted for insertion into the lesion;
determining the major axis and the minor axis of the lesion to be treated, and inserting the distal end of the cryoprobe into said lesion such that the longitudinal axis is substantially aligned with said primary axis;
for lesions having a major axis of 1 cm or less, operating the cryoprobe to create an iceball for a period of 6 minutes, then allowing the iceball to warm for a period of 10 minutes, the, operating the cryoprobe to create the iceball for a period of 10 minutes;
for lesions having a major axis of 1.0 to 1.5 cm, operating the cryoprobe to create an iceball for a period of 8 minutes then allowing the iceball to warm for a period of 10 minutes, the, operating the cryoprobe to create the iceball for a period of 8 minutes;
withdrawing the cryoprobe from the lesion.

5. The method of claim 4 further including visualizing the lesion in the major axis and determining the maximum width dimension to ensure the lesion is less than 1.5 cm and visualizing the lesion in the major axis and determining the maximum radius from the cryoprobe shaft to an outermost edge of the lesion to ensure that this dimension is less than 0.75 cm.

6. The method of claim 5 further including calculating the maximum lesion width dimension to be ablated as twice the maximum radius.

7. The method of claim 5, wherein after the step of operating the cryoprobe for the first cooling period, the diameter of the iceball formed are obtained via ultrasound and if the diameter of the iceball does not exceed the calculated maximum width dimension by 10 mm on all sides, the first cooling time period is extended.

8. The method of claim 5, wherein after the step of operating the cryoprobe for the second cooling period, the diameter of the iceball formed are obtained via ultrasound and if the diameter of the iceball does not exceed the calculated maximum width dimension by 10 mm on all sides, the second cooling time period is extended.
